(19) United States
(12) Patent Application Publication
(10) Pub. No.: US 2006/0099231 A1
(43) Pub. Date: May 11, 2006

(54) Make-up composition for keratin fibres such as eyelashes

(76) Inventors: Valerie De La Poterie, Lailly-En-Val (FR); Karl Pays, Chevilly-Larue (FR); Therese Daubige, Beaumont-En-Val (FR); Patrice Styczen, Gif-sur-Yvette (FR); Yohann Bichon, Maisons-Alfort (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/526,674

(22) PCT Filed: Aug. 6, 2003
(86) PCT No.: PCT/FR03/02479

§ 371 Date: Oct. 11, 2005

(30) Foreign Application Priority Data
Sep. 6, 2002 (FR) 02/11104
Sep. 6, 2002 (FR) 02/11097
Sep. 30, 2002 (FR) 02/12097

(51) Int. Cl. A61K 8/37; A61K 8/31

(52) U.S. Cl. 424/401

(57) ABSTRACT

The invention relates to a cosmetic make-up composition for keratin fibres. The inventive composition is characterised in that it comprises a non-aqueous solvent medium, at least 3 wt.-% of at least one wax in relation to the total weight of the composition, at most 20 wt.-% of water and/or water-soluble solvent in relation to the total weight of the composition. Said composition is further characterised in that it comprises at least 45 wt.-% of dry matter in relation to the total weight of the composition and a plateau modulus of rigidity Gp which is less than or equal to 35,000 Pa.

The present invention relates to making up keratin materials, especially keratin fibers, for instance the eyelashes, the eyebrows and the hair, and more particularly to making up the eyelashes.

The composition according to the invention may be in the form of a mascara, an eyebrow product, an eyeliner or a hair makeup product. The invention relates more especially to a mascara. It may especially be a makeup composition, a composition to be applied over or under a makeup, also known, respectively, as a “top coat” or a “base coat”, or alternatively a composition for treating the eyelashes.

In general, compositions for making up keratin fibers and especially the eyelashes consist of at least one wax or a mixture of waxes dispersed in a liquid phase. It is mainly by means of the amount of wax and of the other nonvolatile ingredients, reflected by the solids content of the composition, that the desired application specificities for the compositions are adjusted, for instance their fluidity, their covering power and/or their curling power, and also their thickening power (also known as charging power or makeup power).

There are in practice essentially two types of mascara formulation, i.e., first, water-based mascaras, known as “cream mascaras”, which are in the form of a wax-in-water water emulsion, and, secondly, anhydrous mascaras or mascaras with a low content of water and/or of water-soluble solvents, known as “waterproof mascaras”, which are formulated in the form of a dispersion of waxes in nonaqueous solvents. It should be noted, however, that certain mascaras in the form of wax-in-water emulsions are also termed “waterproof”. Their water resistance results essentially from the presence of a large amount of latex in their composition. They are also characterized by a low solids content and thus have very little makeup power.

The present invention relates more particularly to the field of keratin fiber makeup compositions containing no water or water-soluble solvent, or having a low content of water and/or of water-soluble solvents, known as “waterproof mascaras”, which are in the form of a dispersion of wax(es) in nonaqueous solvents.

Conventionally, these compositions have a solids content of between 15% and 45% by weight approximately relative to the total weight of the composition.

As mentioned previously, this solids content range is generally unsatisfactory in terms of makeup result. However, if it is desired to increase this solids content beyond this value, a problem of lack of fluidity is rapidly encountered. The makeup composition becomes too thick to apply and also no longer has the deformability required for uniform application over the entire surface of the eyelashes. Moreover, microscopic observation shows that, in this type of composition, the wax particles are generally in the form of aggregates.

The aim of the present invention is, specifically, to propose a makeup or care composition for keratin fibers having a high solids content, which makes it possible especially to obtain a makeup result that is thicker than that obtained with traditional “waterproof” compositions, while at the same time maintaining a consistency that is compatible with the intended makeup use.

One subject of the present invention is thus a cosmetic composition for making up keratin fibers, comprising a nonaqueous solvent medium, at least one wax in a content greater than 3% by weight relative to the total weight of the composition, up to 20% by weight of water and/or of water-soluble solvent relative to the total weight of the composition and having a solids content of greater than 45% by weight relative to the total weight of the composition and a plateau modulus of stiffness Gp of less than or equal to 35 000 Pa.

According to another of its aspects, a subject of the present invention is also a process for preparing a composition as defined above, comprising at least the continuous blending of at least one wax, by passing from a temperature above the melting point of said wax to room temperature with continuous cooling.

A subject of the present invention is also a process for preparing a composition as defined above, comprising at least one step of dispersing at least one wax in the form of particles ranging from 0.5μm to 30μm and in particular ranging from 1 to 20μm in size, in at least one nonaqueous solvent, said solvent being at a temperature below the melting point of said wax in particle form.

The present invention also relates to a process for making up keratin fibers, in which a composition as defined above or as obtained via one of the processes as defined above is applied to said keratin fibers, especially the eyelashes.

Advantageously, the compositions of the invention also have a higher drying speed than standard waterproof compositions, which obviously makes it possible to reduce the time required to perform the makeup process and the risk of transferring makeup from the eyelashes onto the adjacent eyelid. This also makes it possible, where appropriate, to be able to apply several layers of said composition in a satisfactory time and thus to further reinforce the thickening effect of the makeup obtained with these compositions.

### CHARACTERIZATION OF THE SOLIDS

For the purposes of the present invention, the “solids content” denotes the content of nonvolatile matter.

This amount of solids, commonly referred to as the “dry extract” or its abbreviated form E.S., of the compositions according to the invention is measured by heating the sample with infrared rays with a wavelength of from 2μm to 3.5 μm. The substances contained in said compositions that have a high vapor pressure evaporate under the effect of this radiation. Measurement of the weight loss of the sample makes it possible to determine the “dry extract” of the composition. These measurements are performed using a “LP16^{⊙}” commercial infrared desiccator from Mettler.

This technique is fully described in the machine documentation supplied by Mettler.

The measuring protocol is as follows:

About 1 g of the composition is spread onto a metal crucible. After placing the crucible in the desiccator, it is subjected to a nominal temperature of 120° C. for 1 hour. The wet mass of the sample, corresponding to the initial mass and the dry mass of the sample, corresponding to the mass after exposure to the radiation, are measured using a precision balance.

The solids content is calculated in the following manner:
Dry extract=100×(dry mass/wet mass).

The values measured using the protocol described above may differ from the corresponding theoretical values by plus or minus 1%.

The compositions according to the invention are characterized especially by a solids content of greater than 45% by weight, especially ranging from 46% to 80%, in particular from 48% to 70% and more particularly from 50% to 65% by weight, relative to the total weight of the composition.

### RHEOLOGICAL CHARACTERISTICS

The composition in accordance with the invention is further characterized by its viscoelastic behavior, in particular using different Theological parameters.

In general, a material is said to be viscoelastic when, under the effect of shear, it has both the characteristics of a purely elastic material, i.e. capable of storing energy, and the characteristics of a purely viscous material, i.e. capable of dissipating energy.

More particularly, the viscoelastic behavior of the compositions in accordance with the invention may be characterized by its modulus of stiffness G, its elasticity δ and its flow threshold τ_{c}; these parameters are defined especially in the publication “Initiation à la rhéologie [Introduction to Rheology]”, G. Couarraze and J. L. Grossiord, 2nd edition, 1991, published by Lavoisier-Tec 1 Doc.

These parameters are determined by means of measurements performed at 25° C.±0.5° C. using a Haake RheoStress 600® controlled-stress rheometer from the company ThermoRheo, equipped with a stainless-steel rotor with plate/plate geometry, the plate having a diameter of 20 mm and a gap (distance between the lower plate—known as the stator plate—on which the composition is deposited, and the upper plate—known as the rotor plate) of 0.3 mm. The two plates are striated to limit the sliding phenomena to the walls of the plates.

The dynamic measurements are performed by applying a harmonic variation of the stress. In these experiments, the magnitudes of the shear, the shear rate and the stress are low so as to remain within the limits of the linear viscoelastic domain of the material (conditions for evaluating the Theological characteristics of the composition at rest).

The linear viscoelastic domain is generally defined by the fact that the response of the material (i.e. the strain) is at any moment directly proportional to the value of the applied force (i.e. the stress). In this domain, the applied stresses are small and the material undergoes strains without modifying its microscopic structure. Under these conditions, the material is studied “at rest” and nondestructively.

The composition is subjected to a harmonic shear according to a stress τ(t) varying sinusoidally according to a pulse ω(ω=2Πυ, υ being the frequency of the applied shear). The composition thus sheared undergoes a stress τ(t) and responds according to a strain γ(t) corresponding to micro-strains for which the modulus of stiffness varies little as a function of the imposed stress.

The stress τ(t) and the strain γ(t) are defined, respectively, by the following relationships:
r(t)=τ₀ cos(ω·t) γ(t)=γ₀ cos(ω·t−δ)
τ₀ being the maximum amplitude of the stress and γ₀ being the maximum amplitude of the strain. δ is the dephasing angle between the stress and the strain.

The measurements are performed at a frequency of 1 Hz (ν=1 Hz).

The change in the modulus of stiffness G (corresponding to the ratio of τ₀ to γ₀) and in the elasticity δ (corresponding to the dephasing angle of the applied stress relative to the measured strain) as a function of the applied stress τ(t) are thus measured.

The strain of the composition is measured in particular for the stress region in which the variation of the modulus of stiffness G and of the elasticity δ is less than 7% (micro-strain zone), and the “plateau” parameters Gp and δₚ are thus determined. The threshold stress τ_{c} (corresponding to-the minimum force that it is necessary to apply to the composition to cause it to flow) is determined from the curve δ=f (τ) and corresponds to the value of τ for which δ (τ_{c})=1.05 δₚ.

The viscoelastic behavior of the compositions according to the invention is characterized by a plateau modulus of stiffness Gp of less than or equal to 35 000 Pa, especially less than or equal to 30 000 Pa, in particular less than or equal to 28 000 Pa, more particularly less than or equal to 25 000 Pa, or even 20 000 Pa.

The compositions in accordance with the invention may moreover have a flow threshold τ_{c} ranging from 10 Pa to 200 Pa and in particular ranging from 20 Pa to 100 Pa.

### NONAQUEOUS SOLVENT MEDIUM

The composition according to the invention comprises a nonaqueous solvent medium.

This medium is capable of forming a continuous phase and contains, as its name indicates, at least one nonaqueous solvent which is generally a water-insoluble volatile compound that is liquid at room temperature and atmospheric pressure.

For the purposes of the invention, the term “volatile compound” refers to any compound (or nonaqueous medium) capable of evaporating on contact with the skin or the keratin fiber in less than 1 hour, at room temperature and atmospheric pressure. The volatile compound is a volatile cosmetic compound, which is liquid at room temperature, especially having a nonzero vapor pressure, at room temperature and atmospheric pressure, especially having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

In contrast, the term “nonvolatile compound” refers to a compound that remains on the skin or the keratin fiber at room temperature and atmospheric pressure at least for several hours and that especially has a vapor pressure of less than 10⁻³mmHg (0.13 Pa).

The volatile compounds are generally present in a majority proportion in the nonaqueous solvent medium, that is to say they represent more than 50% by weight of said nonaqueous solvent medium. In particular, they may represent at least 60%, more particularly at least 70% and may be up to 100% by weight relative to the total weight of said nonaqueous solvent medium.

The content of water-insoluble volatile compound that is liquid at room temperature is generally less than 55%, and is especially from 10 to 54%, in particular between 15% and 52% and more particularly between 17.5% and 50% by weight relative to the total weight of the composition.

The water-insoluble volatile compound that is liquid at room temperature is in particular a cosmetically acceptable oil or organic solvent. The term “cosmetically acceptable” means a compound whose use is compatible with application to keratin fibers and to the skin.

Needless to say, the nonaqueous solvent medium of the composition according to the invention may comprise a mixture of such compounds.

The volatile oils may be hydrocarbon-based oils, silicone oils and/or fluoro oils, or mixtures thereof.

The term “hydrocarbon-based oil” means an oil mainly containing hydrogen and carbon atoms and possibly oxygen, nitrogen, sulfur and phosphorus atoms. The volatile hydrocarbon-based oils may be chosen from hydrocarbon-based oils containing from 8 to 16 carbon atoms, and especially C₈-C₁₆ branched alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade names “Isopars^{⊙}” or “Permethyl^{⊙}”, branched C₈-C₁₆ esters and isohexyl neopentanoate, and mixtures thereof. Other volatile hydrocarbon-based oils, for instance petroleum distillates, especially those sold under the name “Shell Solt^{⊙}” by the company Shell, may also be used.

Volatile silicones may also be used as volatile oils, for instance volatile linear or cyclic silicone oils, especially those with a viscosity ≦6 centistokes (6×10⁻⁶ m²/s) and especially containing from 2 to 10 silicon atoms, these silicones optionally comprising alkyl or alkoxy groups containing from 1 to 22 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

It is also possible to use volatile organic solvents that are especially fluorinated, such as nonafluoromethoxybutane or perfluoromethylcyclopentane.

According to one particular embodiment of the compositions according to the invention, the water-insoluble volatile compound that is liquid at room temperature is chosen from volatile hydrocarbon-based oils containing from 8 to 16 carbon atoms, and mixtures thereof.

The nonaqueous solvent medium may also comprise at least one water-insoluble nonvolatile compound that is liquid at room temperature, especially at least one nonvolatile oil, which may be chosen in particular from nonvolatile hydrocarbon-based oils and/or silicone oils and/or fluoro oils.

Nonvolatile hydrocarbon-based oils that may especially be mentioned include:

hydrocarbon-based oils of plant origin, such as triglycerides consisting of fatty acid esters of glycerol, the fatty acids of which may have chain lengths ranging from C₄ to C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil and musk rose oil; or alternatively caprylic/capric acid triglycerides, for instance those sold by the company Stearineries Dubois or those sold under the names “Miglyol 810^{⊙}”, “812^{⊙}” and “818^{⊙}” by the company Dynamit Nobel,

synthetic ethers containing from 10 to 40 carbon atoms;

linear or branched hydrocarbons of mineral or synthetic origin, such as petroleum jelly, polydecenes, hydrogenated polyisobutene such as parleam, and squalane, and mixtures thereof;

synthetic esters, for instance oils of formula R₁COOR₂ in which R₁ represents a linear or branched fatty acid residue containing from 1 to 40 carbon atoms and R₂ represents a hydrocarbon-based chain, especially a branched chain, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexylpalmitate, isostearyl isostearate, and alcohol or polyalcohol octanoates, decanoates or ricinoleates, for instance propylene glycol dioctanoate; hydroxylated esters, for instance isostearyl lactate or diisostearyl maleate; and pentaerythritol esters;

fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid and linolenic acid; and mixtures thereof.

The nonvolatile silicone oils that may be used in the composition according to the invention may be nonvolatile polydime-thylsiloxanes (PDMSs), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

The fluoro oils that may be used in the composition of the invention are especially fluorosilicone oils, fluoro polyethers and fluorosilicones as described in document EP-A-847 752.

The content of water-insoluble nonvolatile compound that is liquid at room temperature is generally from 0.01% to 25% by weight and in particular-from 0.1% to 22% by weight relative to the total weight of the composition.

### WATER AND/OR WATER-SOLUBLE SOLVENT

According to the first variant of the invention, the compositions proposed are free of water and of water-soluble solvents.

According to a second variant of the invention, the compositions proposed comprise water and/or at least one water-soluble solvent in a total content of less than or equal to 20% by weight relative to the weight of the composition.

The term “water-soluble solvent” denotes a compound that is liquid at room temperature and miscible with water (water miscibility of greater than 50% by weight at 25° C. and atmospheric pressure).

The water-soluble solvents that may optionally be used in the compositions according to the invention are also generally volatile.

Among the water-soluble solvents that may be used in the compositions according to the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The water and/or the water-soluble solvent(s) may be introduced as such into the formulation according to the invention or may be incorporated therein by means of one or more ingredients constituting said composition. Thus, water may especially be introduced into the composition by means of the introduction of latex or pseudolatex, i.e. an aqueous dispersion of polymer particles.

The presence of water and/or of water-soluble solvent(s) in said compositions may be advantageous for increasing the adhesion of the composition to the eyelashes. Specifically, the larger the amount of nonaqueous solvent, the more slippery the application onto the eyelashes, on account of the mainly “oily” nature of the composition. The partial replacement of the nonaqueous solvent with a water-soluble solvent makes it possible to reduce this effect and thus to increase the adhesion to the eyelashes. The makeup result obtained is then thicker.

In this variant of the invention, the content of water and/or water-soluble solvent(s) may especially be greater than or equal to 0.5%, in particular ranging from 1% to 18% and more particularly from 2% to 15% by weight, relative to the total weight of the composition.

### WAX(ES)

The compositions according to the invention comprise a wax or a mixture of waxes in a content of greater than 3% by weight relative to the total weight of the composition.

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than or equal to 45° C. and in particular greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name “MDSC 2920” by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 10° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at room temperature, of animal, plant, mineral or synthetic origin, and mixtures thereof.

The waxes that may be used in the compositions according to the invention generally have a hardness ranging from 0.01 MPa to 15 MPa, especially greater than 0.05 MPa and in particular greater than 0.1 MPa.

The hardness is determined by measuring the compression force, measured at 20° C. using a texturometer sold under the name “TA-XT2i^{⊙}” by the company Rheo, equipped with a stainless-steel cylindrical spindle 2 mm in diameter, by measuring the change in force (compression force or stretching force) (F) as a function of time, during the following operation:

The spindle is displaced at a speed of 0.1 mm/s and then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.1 mm/s. During the relaxation time, the force (compression force) decreases greatly until it becomes zero, and then, during the withdrawal of the spindle, the force (stretching force) becomes negative and then rises again toward the value 0. The hardness corresponds to the maximum compression force measured between the surface of the spindle and the wax at the moment they come into contact. The value of this force is expressed in MPa.

To measure the hardness, the wax is melted at a temperature equal to the melting point of the wax +20° C. The molten wax is poured into a container 30 mm in diameter and 20 mm deep. The wax is recrystallized at room temperature (25° C.) for 24 hours and is then stored for at least 1 hour at 20° C., before performing the hardness measurement.

As illustrations of waxes that are suitable for the invention, mention may be made especially of hydrocarbon-based waxes, for instance beeswax, lanolin wax, Chinese insect waxes, sumach wax, paraffins, certain polyethylene waxes and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference “Iso-Jojoba-50^{⊙}”, hydrogenated sunflower oil, hydrogenated castor oil, hydrogenated coconut oil, hydrogenated lanolin oil and bis(1,1,1-trimethylolpropane) tetrastearate sold under the name “Hest 2T-4S^{⊙}” by the company Heterene.

Mention may also be made of silicone waxes and fluoro waxes.

The waxes obtained by hydrogenation of castor oil esterified with cetyl alcohol, sold under the names “Phytowax ricin 16L64^{⊙}” and “22L73^{⊙} by the company Sophim may also be used. Such waxes are described in patent application FR-A-2 792 190.

According to one particular embodiment, the compositions according to the invention may comprise at least one “tacky” wax, i.e. a wax with a tack of greater than or equal to 0.7 N.s and a hardness of less than or equal to 3.5 MPa.

Using a tacky wax may especially make it possible to obtain a cosmetic composition that applies easily to keratin fibers, attaches well to the keratin fibers and leads to the formation of a smooth, uniform and thickening makeup result.

The tacky wax used may especially have a tack ranging from 0.7 N.s to 30 N.s, in particular greater than or equal to 1 N.s, especially ranging from 1 N.s to 20 N.s, in particular greater than or equal to 2 N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name “TA-XT2i^{⊙}” by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

Tacky waxes that may be used include a C₂₀-C₄₀ alkyl (hydroxystearyloxy)stearate (the alkyl group containing from 20 to 40 carbon atoms), alone or as a mixture, in particular a C₂₀-C₄₀ alkyl 12-(12′-hydroxystearyloxy)-stearate, of formula (I): 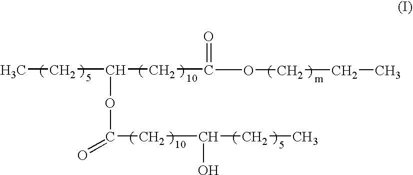
in which m is an integer ranging from 18 to 38, or a mixture of compounds of formula (I).

Such a wax is especially sold under the names “Kester Wax K 82 P^{⊙}” and “Kester Wax K 80 P^{⊙}” by the company Koster Keunen.

The waxes mentioned above generally have a starting melting point of less than 45° C.

According to one particular embodiment of the compositions according to the invention, they may comprise at least one wax with a high starting melting point, i.e. generally greater than or equal to 45° C., in particular greater than or equal to 50° C., or even a very high starting melting point, i.e. generally greater than or equal to 55° C. and in particular greater than or equal to 60° C. The starting melting point of the wax may be measured using a differential scanning calorimeter (DSC), for example the calorimeter sold under the name “MDSC2920^{⊙}” by the company TA Instruments.

The measuring protocol is the same as that described for measuring the melting point.

The starting melting point, denoted hereinbelow by the abbreviation “mpₛₜₐᵣₜ”, of the compound corresponds to the temperature measured when 5% of the heat of fusion is consumed.

As waxes with a starting melting point that is high but less than 50° C., mention may be made especially of montan wax (mpₛₜₐᵣₜ=47.9° C.), ozokerite (mpₛₜₐᵣₜ=46.3° C.) and the wax obtained by catalytic hydrogenation of olive oil esterified with stearyl alcohol, sold under the name “Phytowax Olive 18L57^{⊙}” by the company Sophim (mpₛₜₐᵣₜ=47.4° C.)

As waxes with a melting point of greater than or equal to 50° C. and less than 55° C., mention may be made especially of rice bran wax (mpₛₜₐᵣₜ=51.4° C.), candelilla wax (mpₛₜₐᵣₜ=50° C.) and ouricurry wax (mpₛₜₐᵣₜ=51.4° C.).

As waxes with a very high starting melting point that may be used in the compositions according to the invention, mention may be made especially of carnauba wax (mpₛₜₐᵣₜ=63.5° C.), the waxes obtained by Fisher-Tropsch synthesis (mpₛₜₐᵣₜ=60.5° C.), certain polyethylene waxes such as, especially, those sold under the name “Performalene 655^{⊙}” by the company New Phase Technologies or “Polyethylene AC 540^{⊙}” by the company Honeywell, “Polywax 2000 T-6^{⊙}” by the company Petrolite (mpₛₜₐᵣₜ=125° C.) or “PED 191^{⊙}” and “Epolene N-14^{⊙}” by the company Eastman Kodak (mpₛₜₐᵣₜ=120° C. and 106° C., respectively) and certain monocrystalline waxes such as those sold under the names “Tisco Wax 88^{⊙}” by the company Tisco or “Microwax HW^{⊙}” by the company Paramelt.

As waxes with a very high starting melting point, mention may also be made of waxes obtained by catalytic hydrogenation of animal or plant oils containing linear or branched C₈-C₃₂ fatty chains, such as hydrogenated jojoba oil (mpₛₜₐᵣₜ=63.2° C.) and bis(l,1,1-trimethylol-propane) tetrabehenate sold under the name “Hest 2T-4B^{⊙}” by the company Heterene (mpₛₜₐᵣₜ=61.8° C.).

The content of wax with a high, or even very high, starting melting point in the compositions according to the invention may be particularly high and may especially be greater than or equal to 20% by weight.

In the present invention, it is also possible to use waxes supplied in the form of small particles of about from 0.5 to 30 micrometers, in particular from 1 to 20 micrometers and more particularly from 5 to 10 micrometers in size, which are denoted hereinbelow as “microwaxes”. For the purposes of distinction, the waxes used according to the invention in the form of larger fragments are denoted hereinbelow as “conventional waxes”.

As microwaxes that may be used in the compositions according to the invention, mention may be made of carnauba microwaxes, such as the product sold under the name “MicroCare 350^{⊙}” by the company Micro Powders, synthetic microwaxes, such as that product sold under the name “MicroEase 114S®” by the company Micro Powders, microwaxes consisting of a mixture of carnauba wax and polyethylene wax, such as the products sold under the names “Micro Care 300^{⊙}” and “310^{⊙}” by the company Micro Powders, microwaxes consisting of a mixture of carnauba wax and of synthetic wax, such as the product sold under the name “Micro Care 325®” by the company Micro Powders, polyethylene microwaxes, such as the products sold under the names “Micropoly 200®”, “220®”, “220L®” and “250S®” by the company Micro Powders, and polytetrafluoroethylene micropowders such as the products sold under the names “Microslip 519®” and “519 L®” by the company Micro Powders.

Among the microwaxes mentioned above, some of them, for instance carnauba microwax, the synthetic microwax “MicroEase 114S®” or the microwax consisting of a mixture of carnauba wax and of synthetic wax “MicroCare 325®”, have a starting melting point of greater than or equal to 45° C.

In the composition according to the invention, it is obviously possible to use a mixture of waxes and especially to use one or more conventional waxes, such as, especially, a tacky wax and/or a wax with a starting melting point of greater than or equal to 45° C., and one or more microwaxes.

The composition according to the invention generally contains from 10% to 70% by weight of waxes. In particular, it may contain from 15% to 65%, more particularly from 20% to 60% or even from-25% to 55% by weight of wax(es) relative to the total weight of the composition.

The wax or the mixture of waxes is present in the compositions according to the invention in the form of a dispersion of particles in the nonaqueous solvent medium.

The wax particles may have varied shapes. They may especially be substantially spherical.

Microscopic observation of a sample of the composition, at room temperature, shows a good dispersion of the wax particles in said medium, with little or no aggregation of these particles, or even a distribution of the particles that is substantially identical in all directions.

Polymer(s) that are Soluble in the Nonagueous Solvent Medium and that have at least One Crystallizable Portion

According to one particular embodiment, the composition according to the invention comprises at least one polymer that is soluble in the nonaqueous solvent medium and that has at least one crystallizable portion.

The expression “polymer that is soluble in said nonaqueous solvent medium” means a polymer which, when introduced alone in a solids content at least greater than 0.01% by weight and for an amount corresponding to that envisaged for the desired final composition, is soluble in said nonaqueous solvent medium at room temperature, generally of about 25° C., and under atmospheric pressure (750 mmHg, i.e. 105 Pa).

For the purposes of the present invention, the term “polymer” denotes a compound containing at least two repeating units, especially at least three repeating units, in particular at least ten repeating units, or even at least fifteen repeating units. The polymer in accordance with the invention is generally composed of at least two repeating units of different nature (copolymer). The polymers used in the invention are generally of synthetic origin and are characterized by molar masses ranging from 200 to 1 000 000 g/mol, in particular from 500 to 500 000 g/mol and more particularly from 1000 to 300 000 g/mol.

More specifically, the polymers used in the present invention are copolymers that are dissolved and noncrystalline in the medium at room temperature and necessarily comprise at least one crystallizable portion denoted A and at least one “amorphous” noncrystallizable portion, denoted B.

As a result of this specific structure, they advantageously have both affinity for waxes by virtue of the portion A and affinity for the solvent by virtue of the portion B, and thus participate efficiently in this respect in dispersing the waxes in the nonaqueous solvent medium.

The crystallizable portion of the polymers used in the present invention represents at least 5%, in particular at least 10% and up to 50%, and more particularly from 30% to 50% by weight relative to the total weight of each polymer.

The crystallizable portion A of a copolymer according to the invention may feature a pendent chain linked to the skeleton of said polymer and/or a block directly incorporated into this skeleton and/or at least one end chain. These copolymers may be of any chemical structure: random, block or grafted copolymers and/or dendrimers.

Similarly, the amorphous portion of a copolymer according to the invention may feature a pendent chain linked to the skeleton of said copolymer and/or a block directly incorporated into this skeleton and/or at least one end chain.

For the purposes of the invention, the following terms or expressions have the meanings given hereinbelow:

“crystallizable portion A” means a sequence of at least 5 repeating units which is such that if the homopolymer corresponding to this repeating unit is considered, it would be characterized by a degree of crystallinity of greater than 30%,

“amorphous portion B” means a sequence of at least 5 repeating units which is such that if the homopolymer corresponding to this repeating unit is considered, it would be characterized by a degree of crystallinity of less than 5%, or even zero,

“block incorporated into the skeleton” means a group of atoms consisting of the repetition of a monomer unit, forming part of the main chain of the polymer,

“pendent chain or side group” means a group of atoms forming a branch on the polymer skeleton, and

“end chain” means a group of atoms located on at least one of the ends of the skeleton.

a) Random Copolymers

Random copolymers are preferably polymers with crystallizable pendent chains, which comprise units resulting from the polymerization of at least two monomers, at least one of which has a crystallizable hydrophobic side chain known as X that may be represented by formula II: 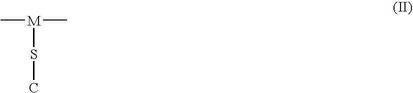
with M representing an atom of the polymer skeleton, S representing a spacer and C representing a crystallizable group.

The crystallizable chains “—S—C” may be aliphatic or aromatic, linear, branched or cyclic and optionally fluorinated or perfluorinated. “S” especially represents a group (CH₂)n or (CH₂CH₂O)n or (CH₂O), which may be linear or branched or cyclic, with n being an integer ranging from 0 to 22. Preferably, “S” is a linear group. Preferably, “S” and “C” are different.

When the crystallizable chains “—S—C” are hydrocarbon-based aliphatic chains, they comprise hydrocarbon-based alkyl chains containing at least 11 carbon atoms and not more than 40 carbon atoms and better still not more than 24 carbon atoms. They are especially aliphatic chains or alkyl chains containing at least 12 carbon atoms, and they are in particular C₁₂-C₂₄ alkyl chains. When they are fluoroalkyl or perfluoroalkyl chains, they contain at least 6 fluorinated carbon atoms and in particular they contain at least 11 carbon atoms, at least 6 of which carbon atoms are fluorinated.

As examples of polymers containing crystallizable pendent chain(s), mention may be made of those comprising units resulting from the polymerization of one or more of the following monomers: (meth)acrylates of saturated alkyls with the alkyl group being C₁₂-C₂₄, perfluoroalkyl (meth)acrylates with a C₁₂-C₁₅ perfluoroalkyl group, N-alkyl(meth)acrylamides with the alkyl group being C₁₂ to C₂₄ with or without a fluorine atom, vinyl or allyl esters containing alkyl or perfluoro(alkyl) chains with the alkyl group being C₁₂ to C₂₄ (with at least 6 fluorine atoms per perfluoroalkyl chain), vinyl ethers containing alkyl or perfluoro(alkyl) chains with the alkyl group being C₁₂ to C₂₄ and at least 6 fluorine atoms per perfluoroalkyl chain, C₁₂ to C₂₄ alpha-olefins such as, for example, octadecene, para-alkylstyrenes with an alkyl group containing from 12 to 24 carbon atoms, and mixtures thereof.

As illustrations of these polymers that may be used in the present invention, mention may be made of copolymers of saturated linear C₁₂ to C₃₀ alkyl (meth)acrylates forming the crystallizable portion A and of linear C₄ to C₁₀ or branched or cyclic and/or unsaturated C₄ to C₃₀ alkyl (meth)acrylates constituting the amorphous portion B.

Among the copolymers of vinyl esters containing linear and saturated C₁₂ to C₃₀ alkyl groups constituting the crystallizable portion A and of vinyl esters containing linear C₄ to C₁₀ or branched or cyclic and/or unsaturated C₄ to C₃₀ alkyl groups constituting the amorphous portion B, mention may be made in particular of copolymers of vinyl acetate and of vinyl stearate or of allyl stearate, such as the copolymer of allyl stearate and of vinyl acetate sold under the name “Mexomer PQ^{⊙}” by the company Chimex.

When the polymers result from a polycondensation, the hydrocarbon-based and/or fluoro crystallizable chains as defined above are borne by a monomer that may be a diacid, a diol, a diamine or a diisocyanate.

b) Block Copolymers

These copolymers consist of at least two types of block of different chemical nature, one of which is crystallizable and constitutes the portion A. In the case of block copolymers, at least one of the amorphous blocks B must be soluble in the medium.

Examples that may be mentioned include:

block copolymers of olefin or of cycloolefin containing a crystallizable chain, for instance those derived from the block polymerization:

of cyclobutene, cyclohexene, cyclooctene, norbornene (i.e. bicyclo[2.2.1]hept-2-ene), 5-methylnorbornene, 5-ethylnorbornene, 5,6-dimethylnorbornene, 5,5,6-trimethylnorbornene, 5-ethylidenenorbornene, 5-phenylnorbonene, 5-benzylnorbornene, 5-vinylnorbornene, 1,4,5,8-dimethano-1,2,3,4,4a,5,8a-octahydro-naphthalene or dicyclopentadiene, or mixtures thereof,

with ethylene, propylene, 1-butene, 3-methyl-1-butene, 1-hexene, 4-methyl-1-pentene, 1-octene, 1-decene or 1-eicosene, or mixtures thereof,

the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

the poly (ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene) -poly(ethylene-propylene)” by P. Richter et al., Macromolecules, 30, 1053-1068 (1997), and

the poly(ethylene)-b-poly(ethylethylene) block copolymers cited in the general article “Crystallization in block copolymers” by I. W. Hamley, Advances in Polymer Science, Vol. 148, 113-137 (1999).

These polymers may have a single crystallizable block or a repetition of crystallizable blocks. In the latter case, these crystallizable blocks may be of identical or different chemical nature.

c) Copolymers Containing Crystallizable End Blocks

Examples that may be mentioned in this category include:

polycondensates of polyamide type resulting from the condensation between (α) at least one acid chosen from dicarboxylic acids containing at least 32 carbon atoms such as dimeric fatty acids, and (β) an alkylenediamine and in particular ethylenediamine, in which the polyamide polymer comprises at least one carboxylic acid end group esterified or amidated with at least one monoalcohol or a monoamine containing from 12 to 30 linear and saturated carbon atoms, and in particular copolymers of ethylenediamine/stearyl dilinoleate, such as the product sold under the name “Uniclear 100 VG ” by the company Arizona Chemical; and

lipophilic polyester polycondensates, the ends of which are esterified with a crystallizable acid or alcohol consisting of a saturated linear C₁₂ to C₃₀ carbon-based chain, and in particular 12-polyhydroxystearic acid, at least one of the ends of which is esterified with stearic acid, such as “Solsperse 21000^{⊙}” sold by the company Avecia.

As further illustrations of the copolymers according to the invention, mention may be made in particular of ethylene/vinyl acetate copolymers, ethylene/maleic anhydride copolymers, hydrogenated butadiene/isoprene block copolymers and ethylene/maleic anhydride/vinyl acetate terpolymers.

The polymer that is soluble in the nonaqueous solvent medium and that has at least one crystallizable portion, or a mixture of such polymers, may be present in the composition according to the invention in a proportion ranging from 0.01% to 30%, especially from 0.1% to 20% and in particular from 1% to 10% by weight relative to the total weight of the composition.

Film-Forming Polymer

According to one particular embodiment, the composition according to the invention may comprise at least one film-forming polymer.

In the present invention, the term “film-forming polymer” means a polymer capable, by itself or in the presence of an auxiliary film-forming agent, of forming a continuous film that adheres to a support and especially to keratin materials.

In the present invention, polymers, generally liposoluble polymers, comprising less than 30% by weight of crystallizable portion under the conditions of the invention and in particular containing none at all, are classified in this category.

Among the film-forming polymers that may be used in the composition of the present invention, mention may be made of synthetic polymers, of free-radical type or of polycondensate type, and polymers of natural origin, and mixtures thereof.

Examples of liposoluble polymers that may be mentioned include copolymers of a vinyl ester (the vinyl group being directly linked to the oxygen atom of the ester group and the vinyl ester containing a saturated, linear or branched hydrocarbon-based radical of 1 to 24 carbon atoms, linked to the carbonyl of the ester group) and of at least one other monomer, which may be a vinyl ester (different than the vinyl ester already present), an alkyl vinyl ether (the alkyl group of which contains from 2 to 24 carbon atoms) or an allylic or methallylic ester (containing a saturated, linear or branched hydrocarbon-based radical of 1 to 24 carbon atoms, linked to the carbonyl of the ester group). These copolymers may be crosslinked using crosslinking agents that may be either of the vinylic type or of the allylic or methallylic type, such as tetra-allyloxyethane, divinylbenzene, divinyl octanedioate, divinyl dodecanedioate and divinyl octadecanedioate.

Examples of these copolymers that may be mentioned include the following copolymers: vinyl acetate/vinyl laurate, vinyl propionate/allyl laurate, vinyl propionate/vinyl laurate, and allyl 2,2-dimethylpentanoate/vinyl laurate.

Liposoluble film-forming polymers that may also be mentioned include liposoluble homopolymers, and in particular those resulting from the homopolymerization of vinyl esters containing from 9 to 22 carbon atoms or of alkyl acrylates or methacrylates, the alkyl radicals containing from 2 to 24 carbon atoms.

Examples of liposoluble homopolymers that may especially be mentioned include: polyvinyl laurate and polylauryl (meth)acrylates, these poly(meth)acrylates possibly being crosslinked using ethylene glycol dimethacrylate or tetraethylene glycol dimethacrylate.

The liposoluble homopolymers and copolymers defined above are known and described especially in patent application FR-A-2 232 303; they may have a weight-average molecular weight ranging from 2000 to 500 000 and in particular from 4000 to 200 000.

As liposoluble film-forming polymers that may be used in the invention, mention may also be made of polyalkylenes and especially C₂ to C₂₀ alkene copolymers, for instance polybutene, alkylcelluloses with a linear or branched, saturated or unsaturated C₁ to C₈ alkyl radical, for instance ethylcellulose and propylcellulose, vinylpyrrolidone (VP) copolymers and especially copolymers of vinylpyrrolidone and of a C₂ to C₄₀ and in particular C₃ to C₂₀ alkene. As examples of VP copolymers that may be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymers.

The film-forming polymer may also be present in the composition in the form of particles dispersed in an aqueous phase or in a nonaqueous solvent phase, which is generally known as a latex or a pseudolatex. The techniques for preparing these dispersions are well known to those skilled in the art.

Aqueous dispersions of film-forming polymer that may be used include the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” by the company Daito Kasey Kogyo; or the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861”, “Sancure 878S” and “Sancure 2060®” by the company Goodrich, “Impranil 85®” by the company Bayer, “Aquamere H-1511®” by the company Hydromer; the sulfopolyesters sold under the brand name “Eastman AQ®” by the company Eastman Chemical Products, vinylic dispersions, for instance “Mexomer PAM” and also acrylic dispersions in isododecane, for instance “Mexomer PAP” by the company CHIMEX.

The film-forming polymer may be present in the composition according to the invention in a solids content ranging from 0.1% to 30% by weight, in particular from 0.5% to 25% by weight and more particularly from 1% to 20% by weight relative to the total weight of the composition.

The composition according to the invention may comprise a plasticizer to promote the formation of a film with the film-forming polymer. Such a plasticizer may be chosen from any compound known to those skilled in the art as being capable of satisfying the desired function.

Dyestuff

The composition according to the invention may also comprise at least one dyestuff, for instance pulverulent dyes, liposoluble dyes and water-soluble dyes.

The pulverulent dyestuffs may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in a content ranging from 0.01% to 30% by weight relative to the total weight of the composition.

Fillers

The composition according to the invention may also comprise at least one filler.

The fillers may be chosen from those that are well known to persons skilled in the art and commonly used in cosmetic compositions. The fillers may be mineral or organic, and lamellar or spherical. Mention may be made of talc, mica, silica, kaolin, polyamide powders, for instance the Nylon® sold under the trade name “Orgasol®” by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name “Expancel®” by the company Nobel Industrie, acrylic powders, such as those sold under the name “Polytrap®” by the company Dow Corning, polymethyl methacrylate particles and silicone resin microbeads (for example “Tospearls®” from Toshiba), precipitated calcium carbonate, magnesium carbonate and magnesium hydrocarbonate, hydroxyapatite, hollow silica microspheres (“Silica Beads®” from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

The fillers may represent from 0.1% to 25% and in particular from 1% to 20% by weight relative to the total weight of the composition.

The composition of the invention may also comprise any cosmetically acceptable additive chosen in particular from those usually used in cosmetics, such as antioxidants, preserving agents, fragrances, neutralizers, plasticizers, thickeners or gelling agents, fibers and cosmetic active agents, and mixtures thereof.

The gelling agents that may be used in the compositions according to the invention are generally lipophilic and may be organic or mineral, and polymeric or molecular.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:

trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references “Aerosil R812®” by the company Degussa, and “Cab-O-Sil TS-530®” by the company Cabot;

dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995). They are sold, for example, under he references “Aerosil R972®” and “Aerosil R974®” by he company Degussa, and “Cab-O-Sil TS-610®” and “Cab-O-Sil TS-720®” by the company Cabot.

The hydrophobic fumed silica preferably has a-particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC₅₅₆®, SR 5CYC gel®, SR DMF 10 gel® and SR DC 556 gel® from Grant Industries and SF 1204® and JK 113® from General Electric; ethylcellulose, for instance those sold under the name Ethocel by Dow Chemical and galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof. Block copolymers of “diblock” or “triblock” type, of the polystyrene/polyisoprene or polystyrene/polybutadiene type such as the products sold under the name “Luvitol HSB®” by the company BASF, of the polystyrene/copoly(ethylene-propylene) type such as the products sold under the name “Kraton®” by the company Shell Chemical Co., or of the polystyrene/copoly(ethylene-butylene) type.

Among the gelling agents that may be used in the compositions according to the invention, mention may also be made of fatty acid esters of dextrin, such as dextrin palmitates, especially the products sold under the name “Rheopearl TL®” or “Rheopearl KL®” by the company Chiba Flour.

The composition according to the invention may also comprise fibers to allow an improvement in the lengthening effect.

The term “fiber” should be understood as meaning an object of length L and diameter D such that L is very much greater than D, D being the diameter of the circle in which the cross section of the fiber is inscribed. In particular, the ratio L/D (or shape factor) is chosen in the range from 3.5 to 2 500, preferably from 5 to 500 and in particular from 5 to 150.

The fibers that may be used in the composition of the invention may be mineral or organic fibers of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibers have a length ranging from 1μm to 10 mm, preferably from 0.1 mm to 5 mm and better still from 1 mm to 3.5 mm. Their cross section may be within a circle of diameter ranging from 2 nm to 500 μm, preferably ranging from 100 nm to 100μm and better still from 1μm to 50 μm. The weight or yarn count of the fibers is often given in denier or decitex, and represents the weight in grams per 9 km of yarn. In particular, the fibers according to the invention may have a yarn count chosen in the range from 0.15 to 30 denier and better still from 0.18 to 18 denier.

The fibers can be those used in the manufacture of textiles, and in particular silk fiber, cotton fiber, wool fiber, flax fiber, cellulose fiber extracted in particular from wood, from plants or from algae, rayon fiber, polyamide (Nylon®) fiber, viscose fiber, acetate fiber, in particular rayon acetate fiber, poly(p-phenyleneterephthalamide) (or aramid) fiber, in particular Kevlar® fiber, acrylic polymer fiber, in particular polymethyl methacrylate fiber or poly(2-hydroxyethyl methacrylate) fiber, polyolefin fiber and in particular polyethylene or polypropylene fiber, glass fiber, silica fiber, carbon fiber, in particular in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers such as those mentioned above, for instance polyamide/polyester fibers.

The fibers used in surgery may also be used, for instance the resorbable synthetic fibers prepared from glycolic acid and caprolactone (Monocryl® from Johnson & Johnson); resorbable synthetic fibers of the type which is a copolymer of lactic acid and of glycolic acid (Vicryl® from Johnson & Johnson); polyterephthalic ester fibers (Ethibond® from Johnson & Johnson) and stainless steel threads (Acier® from Johnson & Johnson).

Moreover, the fibers may be treated or untreated at the surface, and coated or uncoated. As coated fibers that may be used in the invention, mention may be made of polyamide fibers coated with copper sulfide to give an anti-static effect (for example R-STAT® from Rhodia) or another polymer enabling a particular organization of the fibers (specific surface treatment) or surface treatment including color/hologram effects (Lurex® fiber from Sildorex, for example)

In particular, fibers of synthetic origin and in particular organic fibers, such as those used in surgery, are used. Water-insoluble fibers may advantageously be used.

The fibers that may be used in the composition according to the invention may in particular be polyamide fibers, cellulose fibers, poly(p-phenylene-terephthalamide) fibers or polyethylene fibers. Their length (L) may range from 0.1 mm to 5 mm and in particular from 0.25 mm to 1.6 mm, and their mean diameter may range from 1μm to 50μm. In particular, the polyamide fibers sold by Etablissements P. Bonte under the name “Polyamide 0.9 Dtex 3 mm®”, having a mean diameter of 6μm, a yarn count of about 0.9 dtex and a length ranging from 0.3 mm to 5 mm, may be used. Cellulose (or rayon) fibers with a mean diameter of 50μm and a length ranging from 0.5 mm to 6 mm may also be used, for instance those sold under the name “Natural rayon flock fiber RC1BE-N003-M04®” by the company Claremont Flock. Polyethylene fibers, for instance those sold under the name “Shurt Stuff 13 099 F®” by the company Mini Fibers, may also be used.

The composition according to the invention may also comprise “rigid” fibers, as opposed to the fibers mentioned above, which are not rigid fibers.

The rigid fibers, which are initially substantially straight, when placed in a dispersing medium, do not undergo a substantial change in shape, which is reflected by the angular condition defined below, reflecting a shape that may be described as still substantially straight and linear. This angle condition reflects the stiffness of the fibers, which it is difficult to express by another parameter for objects that are as small as the rigid fibers.

The stiffness of the fibers is reflected by the following angular condition: advantageously, at least 50%, in particular at least 75% and more particularly at least 90%, in numerical terms, of the fibers are such that the angle formed between the tangent to the longitudinal central axis of the fiber and the straight line connecting said end to the point on the longitudinal central axis of the fiber corresponding to half the length of the fiber is less than 150, and the angle formed between the tangent to the longitudinal central axis of the fiber at a point half way along the fiber and the straight line connecting one of the ends to the point on the longitudinal central axis of the fiber corresponding to half the length of the fiber, is less than or equal to 15° for the same fiber length ranging from 0.8 mm to 5 mm, in particular ranging from 1 mm to 4 mm, more particularly ranging from 1 mm to 3 mm, or even 2 mm.

Advantageously, the angle mentioned above is measured at the two ends of the fiber and at a point half way along the fiber; in other words, three measurements are taken in this case and the average of the measured angles is less than or equal to 15°.

The tangent, at any point on the fiber, especially forms an angle of less than 15°.

In the present patent application, the angle formed by the tangent at a point on the fiber is the angle formed between the tangent to the longitudinal central axis of the fiber at said point on the fiber and the straight line connecting the end of the fiber that is closest to said point to the point on the longitudinal central axis of the fiber corresponding to half the length of the fiber.

Generally, the rigid fibers that may be used in the composition according to the invention have the same or substantially the same fiber length.

More specifically, when a medium in which are dispersed the rigid fibers to a fiber concentration of 1% by weight is observed by microscope, with an objective lens allowing a magnification of 2.5 and with full-field vision, a numerical majority of the rigid fibers, i.e. at least 50% numerically of the rigid fibers, preferably at least 75% numerically of the rigid fibers and better still at least 90% numerically of the rigid fibers, must satisfy the angular condition defined above. The measurement leading to the angle value is performed for the same length of fibers, this length being in the range from 0.8 mm to 5 mm, in particular from 1 to 4 mm, more particularly from 1 to 3 mm, or even 2 mm.

The medium in which the observation is performed is a dispersing medium that ensures good dispersion of the rigid fibers, for example water or an aqueous gel of clay or of associative polyurethane. A direct observation of the composition containing the rigid fibers may even be performed. A sample of the prepared composition or dispersion is placed between a slide and cover slip for observation by microscope with an objective lens allowing a magnification of 2.5 and with full-field vision. Full-field vision allows the fibers to be viewed in their entirety.

The rigid fibers may be chosen from fibers of a synthetic polymer chosen from polyesters, polyurethanes, acrylic polymers, polyolefins, polyamides, in particular nonaromatic polyamides, and aromatic polyimideamides.

Examples of rigid fibers that may be mentioned include:

polyester fibers, such as those obtained by chopping yarns sold under the names Fiber 255-100-R11-242T Taille 3 mm (eight-lobed cross section)®, Fiber 265-34-R11-56T Taille 3 mm (round cross section)® and Fiber Coolmax 50-34-591 Taille 3 mm (four-lobed cross section)® by the company Dupont de Nemours;

polyamide fibers, such as those sold under the names Trilobal Nylon 0.120-1.8 DPF®; Trilobal Nylon 0.120-18 DPF®; Nylon 0.120-6 DPF by the company Cellusuede Products; or obtained by chopping yarns sold under the name Fiber Nomex Brand 430 Taille 3 mm® by the company Dupont de Nemours;

polyimideamide fibers, such as those sold under the names “Kermel®” and “Kermel Tech®” by the company RHODIA;

poly(p-phenyleneterephthalamide) (or aramide) sold especially under the name Kevlar® by the company Dupont de Nemours;

fibers with a multilayer structure comprising alternating layers of polymers chosen from polyesters, acrylic polymers and polyamides, such as those described in documents EP-A-6 921 217, EP-A-686 858 and U.S. Pat. No. 5 472 798. Such fibers are sold under the names “Morphotex®” and “Teijin Tetron Morphotex®” by the company Teijin.

Rigid fibers that are particularly preferred are aromatic polyimideamide fibers.

Polyimideamide yarns or fibers that may be used for the compositions of the invention are described, for example, in the document from R. Pigeon and P. Allard, Chimie Macromoleculaire Appliquee, 40/41 (1974), pages 139-158 (No. 600), or in documents U.S. Pat. No. 3 802 841, FR-A-2 079 785, EP-A1-0 360 728 and EP-A-0 549 494, to which reference may be made.

The preferred aromatic polyimideamide fibers are polyimideamide fibers comprising repeating units of formula:

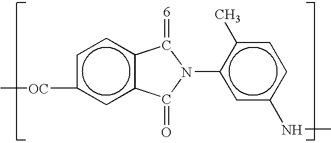

obtained by polycondensation of tolylene disocyanate and trimellitic anhydride.

The fibers may be present in the composition according to the invention in a content ranging from 0.01% to 10% by weight, in particular from 0.1% to 5% by weight and more particularly from 0.3% to 3% by weight relative to the total weight of the composition.

As cosmetic active agents that may be used in the compositions according to the invention, mention may be made especially of emollients, moisturizers, vitamins and screening agents, in particular sunscreens.

Needless to say, a person skilled in the art will take are to select the optional additional additives and/or he amount thereof such that the advantageous properties of the composition according to the invention are not, or are not substantially, adversely affected by the envisaged addition.

### PREPARATION PROCESS

The process for preparing the compositions according to the invention depends in particular on the nature of the wax(es) used. It depends in particular on whether the waxes are of conventional type or of microwax type as defined above. For the conventional waxes, it furthermore depends on the starting melting point of said wax.

In a first variant, the wax(es) used is (are) of conventional type and have a starting melting point of less than 45° C.

In such a case, the compositions of the invention may be prepared in a standard manner by heating the waxes until they have completely melted and then introducing them into a volatile nonaqueous solvent. The mixture thus obtained is subjected to mechanical stirring until it has cooled to room temperature. When the composition comprises a polymer that is soluble in the nonaqueous solvent medium and that has a crystallizable portion, it is generally introduced with the volatile nonaqueous solvent, but may also be introduced subsequent thereto.

The water and/or the water-soluble solvent(s) and the optional additional ingredients may be introduced into the starting materials or, optionally, during the cooling or into the finished composition.

In a second variant, the wax(es) used is (are) of conventional type. Their starting melting point may be less than, equal to or greater than 45° C.

In such a variant, the keratin fiber makeup compositions are generally obtained by heating the wax or a mixture of several waxes to a temperature above the melting point of the wax that has the highest melting point, until they have completely melted, followed by blending and continuous cooling to room temperature.

The nonaqueous solvent may be added during the blending or prior to this operation.

It appears to be that blending the composition instead of stirring it according to a conventional process promotes the crystallization of the wax in the form of fine crystals forming small particles. It also appears that this blending breaks up any particle aggregates formed, leading, in the finished composition, to a substantially homogeneous dispersion of small wax particles.

The blending operation may be performed, for example, in a roll mill comprising two counter-rotating rolls between which is fed the paste, or more advantageously in a continuous twin-screw blender, which allows a paste of very consistent quality to be reproducibly obtained.

The conditions under which the blending operation may be performed are described in patent application FR 94/00756, the content of which is incorporated into the present patent application by reference.

When the composition comprises water and/or a water-soluble solvent and optional additional ingredients, these may be introduced into the starting materials or, optionally, in the course of the blending during the cooling or into the finished composition.

In particular, when the composition comprises at least one polymer that is soluble in the nonaqueous solvent and that has a crystallizable portion, this polymer is introduced in particular prior to the blending operation, separately or along with the nonaqueous solvent.

This method for preparing the compositions in particular has the advantage of allowing the incorporation of heat-sensitive compounds, for instance certain active agents, given that it allows them to be introduced at a temperature that is compatible with their stability and by virtue of the short residence time in the blender.

In a third variant of the invention, the wax(es) used is (are) microwaxes as defined above.

As a result of its formulation in the form of particles, such a wax may be used directly at a temperature below its melting point. In other words, in this particular embodiment of preparation of the compositions according to the invention, the microwax particles are dispersed directly in the continuous phase, rather than forming them therein via melting/recrystallization steps.

This wax dispersion step may be performed in particular at a temperature below the melting point of the wax and especially at room temperature, which is, of course, advantageous in terms of ease of implementation of the preparation process.

The nonaqueous solvent is chosen from those defined above. In this particular preparation process embodiment, the water and/or the water-soluble solvent(s) and/or the additional ingredients as defined above may be added, depending on the case, either into the starting materials or into the finished composition.

In a fourth variant of the invention, the process for preparing the compositions involves both at least one conventional wax and at least one microwax as defined above. In such a case, the conventional wax or the mixture of conventional waxes is generally introduced first, melted in the nonaqueous solvent, where appropriate as a mixture with at least one polymer that is soluble in said solvent and that has a crystallizable portion, and the mixture thus obtained is then stirred or blended while cooling. The microwax or the mixture of microwaxes is introduced only when the temperature of the mixture containing the conventional wax is below the melting point of said microwax or below the melting point of the microwax of the microwax mixture that has the lowest melting point, and especially at room temperature.

In this case also, the water and/or the water-soluble solvent(s) and the additional ingredients may be added, depending on the case, either into the starting materials or into the finished compositions, or else, when the composition is blended, during the cooling operation.

A subject of the present invention is also a process for making up keratin fibers, in which a composition as defined above is applied to said keratin fibers, especially the eyelashes.

The compositions of the invention may in particular be applied to the eyelashes using a brush or a comb.

The thickening effect of the makeup, using the composition of the invention, may moreover be reinforced most particularly by selecting the device for applying said composition.

In the present case, it is particularly advantageous, in the case of making up She eyelashes, to apply said composition with a makeup brush as described in patents FR 2701198, FR 2605505, EP 792603 and EP 663161.

The examples that follow are given as nonlimiting illustrations of the invention.

Composition Preparation Protocol

a. Process for Preparing Compositions Comprising only Waxes in Microparticle Form

The dyestuffs and the gelling agent are dispersed with stirring in at least one nonaqueous solvent, optionally as a mixture with at least one polymer that is soluble in said nonaqueous solvent and that has at least one crystallizable portion, and in such a case the mixture having been preheated to a temperature of 45° C. and then cooled to room temperature. The wax in microparticle form and, where appropriate, the remaining ingredients of the composition are then added, with stirring.

The water and/or the water-soluble solvent(s) are in particular gradually dispersed with stirring.

b. Preparation of Compositions Comprising both Conventional Waxes and Waxes in Microparticle Form

The dyestuffs and the gelling agent are dispersed with stirring in at least one nonaqueous solvent, optionally as a mixture with at least one polymer that is soluble in said nonaqueous solvent and that has at least one crystallizable portion, heated to a temperature of 45° C. and then cooled to room temperature. The mixture obtained is then heated to 45° C., after which the mixture of conventional waxes preheated until completely melted is gradually added. The mixture thus obtained is allowed to cool to room temperature with stirring. The wax in microparticle form and, where appropriate, the remaining ingredients of the composition are then added.

The water and/or the water-soluble solvent(s) are in particular gradually dispersed with stirring.

c. Preparation of the Compositions using a Continuous Twin-Screw Blender

The preparation is performed in a continuous twin-screw blender such as the “BC-21” model from the company Clextral, and takes place under the following conditions:
inlet temperature: 100° C.
outlet temperature: 20° C.
flow rate=3 kg/h
screw speed: 600 rpm.

The premelted waxes are introduced into the top of the blender at the same time as the nonaqueous solvent and the other ingredients, and the mixture is then cooled under continuous twin-screw blending down to the outlet temperature.

Measurement of the Physical Characteristics

The measurement of the solids content is performed according to the protocol described previously.

The Theological measurements were performed according to the protocols described previously, using a “Haake RheoStress 600®” controlled-stress rheometer under the following conditions:

measuring temperature: 25° C.,

steady stage of 180 seconds at 25° C. before starting the measuring,

stressed sweep from 1 to 10 000 Pa,

measuring frequency: 1 Hz.

### EXAMPLES 1, 2 AND 3

Three waterproof mascaras having the compositions given in table I below were prepared according to the process described in b) (in this table, the amounts indicated are weight percentages and are expressed relative to the total weight of the composition):

TABLE I

| | Example | Example | Example |
|---|---|---|---|
| | 1 | 2 | 3 |
| Beeswax | 8.67 | 8.67 | 8.67 |
| Carnauba microwax (“MicroCare | 24.2 | 24.2 | 24.2 |
| 350 ®” from Micro Powders) | | | |
| Synethetic microwax (“MicroEase | 2.02 | 2.02 | 2.02 |
| 114S ®” from Micro Powders) | | | |
| Polyvinyl laurate | 0.66 | 0.66 | 0.66 |
| (“Mexomer PP ®” from Chimex) | | | |
| Preserving agent | 0.2 | 0.2 | 0.2 |
| Colorant | 5.7 | 5.7 | 5.7 |
| Bentonite | 3.6 | 3.6 | 3.6 |
| Propylene carbonate | 1.18 | 1.18 | 1.18 |
| Allyl stearate/vinyl acetate copolymer | 2 | *** | *** |
| (“Mexomer PQ ®” from Chimex) | | | |
| Ethylenediamine/stearyl dilinoleate | *** | 2 | *** |
| copolymer (“Uniclear 100 VG ®” | | | |
| from Arizona Chemical) | | | |
| C₁₀-C₃₀ alkyl acrylate polymer | *** | *** | 2 |
| (from Landec) | | | |
| Isododecane | 51.8 | 51.8 | 51.8 |

Various characteristics of these compositions were studied in vitro according to the protocols described previously.

The results are given in table II below:

TABLE II

| | Characteristics | | | |
|---|---|---|---|---|
| Compositions | [wax] (% m) | D.E. (% m) | [Gp] (Pa) | τc (Pa) |
| Example 1 | 34.9 | 48.5 | 4500 | 40 |
| Example 2 | 24.9 | 48.2 | 2300 | 30 |
| Example 3 | 34.9 | 48.5 | 20 000 | 100 |

The compositions obtained thus have particularly high solids contents combined with a plateau modulus of stiffness that is low enough to allow them to be used under satisfactory conditions.

### EXAMPLE 4

A waterproof mascara having the composition below was prepared according to process b):

| | | |
|---|---|---|
| Beeswax | 8.67 | g |
| Carnauba Microwax | 24.20 | g |
| (“MicroCare 350 ®” from Micro Powders) | | |
| Synthetic Microwax | 2.02 | g |
| (“MicroEase 114S ®” from Micro Powders) | | |
| Polyvinyl laurate | 0.66 | g |
| (“Mexomer PP ®” from Chimex) | | |
| Preserving agent | 0.20 | g |
| Dye | 5.70 | g |
| Bentonite | 3.60 | g |
| Propylene carbonate | 1.18 | g |
| Allyl stearate/vinyl acetate copolymer | 6.50 | g |
| (“Mexomer PQ ®” from Chimex) | | |
| Isododecane | 47.27 | g |

Various characteristics of this composition were studied in vitro according to the protocols described previously. The results are given in table III below:

TABLE III

| | Characteristics | | | |
|---|---|---|---|---|
| Compositions | [wax] (% m) | D.E. (% m) | [Gp] (Pa) | τc (Pa) |
| Example 4 | 34.9 | 53.0 | 1 000 | 9 |

The composition obtained thus has a very high solids content (greater than 50%) having a particularly low modulus of stiffness (1000 Pa).

### EXAMPLES 5 AND 6

Two waterproof mascaras having the compositions respectively presented in table IV below were prepared 5 according to process a) (in this table, the amounts indicated are in weight percentages and are expressed relative to the total weight of the composition):

TABLE IV

| | Example 5 | Example 6 |
|---|---|---|
| Carnauba Microwax | 45 | *** |
| (“MicroCare 350 ®” from Micro Powders) | | |
| Synthetic Microwax | *** | 45 |
| (“MicroEase 114S ®” from Micro Powders) | | |
| Dye | 6 | 6 |
| Bentonite | 3.6 | 3.6 |
| Propylene carbonate | 1.18 | 1.18 |
| Allyl stearate/vinyl acetate copolymer | 6 | 6 |
| (“Mexomer PQ ®” from Chimex | | |
| Isododecane | 38.22 | 38.22 |

The various characteristics of these compositions were studied in vitro according to the protocols described previously.

The results are given in table V below:

TABLE V

| | Characteristics | | | |
|---|---|---|---|---|
| Compositions | [wax] (% m) | D.E. (% m) | [Gp] (Pa) | τc (Pa) |
| Example 5 | 45 | 61.8 | 3 000 | 40 |
| Example 6 | 45 | 61.8 | 160 | 25 |

It is found that the compositions obtained have solids contents of greater than 60% while at the same time having low modulus of stiffness values and thus satisfactory flexibility.

These formulations apply to the eyelashes particularly well and lead to a makeup result that gives substantial thickening of the eyelashes.

### EXAMPLES 7 TO 10

Four waterproof mascaras were prepared according to the process described in b) by mixing together the ingredients given in table VI below (in this table, the amounts indicated are in weight percentages and are expressed relative to the total weight of the composition).

TABLE VI

| | Example | Example | Example | Example |
|---|---|---|---|---|
| | 7 | 8 | 9 | 10 |
| Beeswax | 8.67 | 8.67 | 13 | 13 |
| Carnauba microwax | 12.2 | 16.2 | 18.37 | 18.37 |
| (“MicroCare 350 ®” | | | | |
| from Micro Powders) | | | | |
| Synthetic microwax | 6.52 | 6.52 | 6.52 | 6.52 |
| (“MicroEase 114S ®” | | | | |
| from Micro Powders) | | | | |
| Polyvinyl laurate | 0.66 | 0.66 | *** | *** |
| (“Mexomer PP ®” | | | | |
| from Chimex) | | | | |
| Preserving agent | 0.2 | 0.2 | 0.2 | 0.2 |
| Dye | 5.7 | 5.7 | 5.7 | 5.7 |
| Modified hectorite | 3.6 | 3.6 | 3.6 | 3.6 |
| (“Bentone 8V ®” | | | | |
| from Elementis) | | | | |
| Propylene carbonate | 1.18 | 1.18 | 1.18 | 1.18 |
| Vinylpyrrolidone/1- | *** | 5 | *** | *** |
| eicosene copolymer | | | | |
| (“Antaron V | | | | |
| 220 ®” from ISP) | | | | |
| Allyl stearate/vinyl | 6.5 | 6.5 | 6.5 | 6.5 |
| acetate copolymer | | | | |
| (“Mexomer PQ ®” | | | | |
| from Chimex) | | | | |
| Ethylenediamine/stearyl | 3 | 3 | 1.5 | 1.5 |
| dilinoleate copolymer | | | | |
| (“Uniclear 100 VG ®” | | | | |
| from Arizona Chemical) | | | | |
| Vinyl acetate/vinyl | 3 | 3 | *** | *** |
| t-butyl-benzoate/crotonic | | | | |
| acid copolymer as an | | | | |
| aqueous dispersion | | | | |
| containing 26.3% AM | | | | |
| (“Mexomer PAM ®” | | | | |
| from Chimex) | | | | |
| Ethyl acrylate/methyl | 9 | *** | *** | *** |
| methacrylate copolymer | | | | |
| (80/20) as an aqueous | | | | |
| dispersion containing | | | | |
| 50% AM (“Daitosol | | | | |
| 5000 AD ®” from | | | | |
| Daito) | | | | |
| Water | *** | *** | 7.32 | *** |
| Isododecane | 39.77 | 39.77 | 36.11 | 43.43 |

The compositions of Examples 7 and 8 comprise water in respective contents of 6.5% and 2.1% by weight relative to the total weight of the composition, the water being derived from latices used, i.e. the “Mexomer PAM®” from the company Chimex and the “Daitosol 5000 AD®” from the company Daito.

Various in vitro characteristics of these compositions were studied according to the protocols described previously.

The results are given in Table VII below.

TABLE VII

| | Characteristics | | | |
|---|---|---|---|---|
| Compositions | [wax] (% m) | D.E. (% m) | [Gp] (Pa) | τc (Pa) |
| Example 7 | 27.4 | 53 | 6000 | 53.7 |
| Example 8 | 27.4 | 57.5 | 7000 | 60 |
| Example 9 | 37.9 | 56.5 | 13 000 | 70 |
| Example 10 | 37.9 | 56.8 | 2800 | 20 |

### EXAMPLE 11

A waterproof mascara having the composition below was prepared according to the process described in c):

| | | |
|---|---|---|
| Tacky wax (“Kester Wax K 82 P ®” | 32 | g |
| from the company Koster Keunen) | | |
| Dextrin palmitate (“Rheopearl KL ®” | 5.32 | g |
| from Chiba Flour) | | |
| Vinyl acetate/allyl stearate copolymer (65/35) | 2.2 | g |
| (“Mexomer PQ ®” from Chimex) | | |
| Polyvinyl laurate | 0.75 | g |
| (“Mexomer PP ®” from Chimex) | | |
| 12-Hydroxystearic acid oligomer stearate | 0.10 | g |
| (“Solsperse 21000 ®” from Avecia) | | |
| Silica | 10 | g |
| Talc | 0.84 | g |
| Pigments | 4.62 | g |
| Preserving agents | qs | |
| Nondenatured 96° ethyl alcohol | 3 | g |
| Isododecane | 40.96 | g |

The various in vitro characteristics of this composition were studied according to the protocols described previously.

The results are given in Table VIII below.

TABLE VIII

| | Characteristics | | | |
|---|---|---|---|---|
| Compositions | [wax] (% m) | D.E. (% m) | [Gp] (Pa) | τc (Pa) |
| Example 11 | 32 | 56.0 | 13 000 | 90 |

The composition obtained thus also has a particularly high solids content, linked to a modulus of stiffness that is low enough-to allow its use under satisfactory conditions.

The mascara applies easily to the eyelashes and allows a thick and nontacky makeup result to be obtained on the eyelashes: the eyelashes are well separated.

### EXAMPLE 12

A waterproof mascara having the composition below was prepared according to the process described in b):

| | | |
|---|---|---|
| Microcrystalline wax (“Microwax HW ®” from | 30 | g |
| Paramelt) | | |
| Pigments | 9.24 | g |
| Rice starch | 1.68 | g |
| Vinyl acetate/allyl stearate copolymer (“Mexomer | 4.42 | g |
| PQ ®” from Chimex) | | |
| Polyvinyl laurate (“Mexomer PP ®” from Chimex) | 1.5 | g |
| Modified hectorite (“Bentone 38V ®” from | 0.63 | g |
| Elementis) | | |
| Propylene carbonate | 0.21 | g |
| Ethyl alcohol | 3.0 | g |
| Stearate of 12-hydroxystearic acid oligomer | 0.2 | g |
| (“Solsperse 21000 ®” from Avecia) | | |
| Preserving agent | 0.4 | g |
| Isododecane | 48.72 | g |

This composition has a solids content of 48.28% by weight relative to the total weight of the composition.

1. A cosmetic composition for making up keratin fibers, characterized in that it comprises a nonaqueous solvent medium, at least one wax in a content of greater than 3% by weight relative to the total weight of the composition, up to 20% by weight of water and/or of water-soluble solvent relative to the total weight of the composition and in that it has a solids content of greater than 45% by weight relative to the total weight of the composition and a plateau modulus of stiffness Gp of less than or equal to 35 000 Pa.

2. The composition as claimed in claim 1, characterized in that said solids content is from 46% to 80%, in particular from 48% to 70% and more particularly from 50% to 65% by weight, relative to the total weight of the composition.

3. The composition as claimed in claim 1 or 2, characterized in that said plateau modulus of stiffness Gp is less than or equal to 30 000 Pa, in particular less than or equal to 28 000 Pa, more particularly less than or equal to 25 000 Pa or even 20 000 Pa.

4. A composition as claimed in any one of the preceding claims, characterized in that the nonaqueous solvent medium comprises at least one water-insoluble volatile compound that is liquid at room temperature.

5. The composition as claimed in either of the preceding claims, characterized in that said volatile compound represents more than 50% by weight of said nonaqueous solvent medium.

6. The composition as claimed in either of claims 4 and 5, characterized in that said water-insoluble volatile compound is chosen from hydrocarbon-based oils, silicone oils and/or fluoro oils, and organic solvents, and mixtures thereof, and in particular from hydrocarbon-based oils containing from 8 to 16 carbon atoms, and mixtures thereof.

7. The composition as claimed in any one of claims 4 to 6, characterized in that said water-insoluble volatile compound is present in the composition in a content of less than 55%, especially between 10% and 54%, in particular between 15% and 52% and more particularly between 17.5 and 50% by weight relative to the total weight of the composition.

8. The composition as claimed in any one of claims 4 to 7, characterized in that the nonaqueous solvent medium also comprises at least one nonvolatile oil.

9. The composition as claimed in any one of the preceding claims, characterized in that said composition is free of water and of water-soluble solvent.

10. The composition as claimed -in any one of claims 1 to 8, characterized in that the total content of water and/or of water-soluble solvent(s) is greater than or equal to 0.5%, in particular from 1% to 18% and more particularly from 2% to 15% by weight relative to the total weight of the composition.

11. The composition as claimed in claim 10, characterized in that said water-soluble solvent is chosen from lower monoalcohols containing from 1 to 5 carbon atoms, glycols containing from 2 to 8 carbon atoms, C₃ and C₄ ketones and C₂ to C₄ aldehydes.

12. The composition as claimed in any one of the preceding claims, characterized in that said wax is chosen from waxes that are solid and rigid at room temperature, with a melting point of greater than or equal to 30° C., in particular greater than or equal to 45° C. and especially greater than or equal to 55° C.

13. The composition as claimed in any one of the preceding claims, characterized in that the wax is chosen from hydrocarbon-based waxes, for instance beeswax, lanolin wax, Chinese insect waxes, sumach wax, paraffins, polyethylene waxes, waxy copolymers, and esters thereof; the waxes obtained by catalytic hydrogenation of animal or plant oils containing linear or branched C₈-C₃₂ fatty chains, for instance trans-isomerized partially hydrogenated jojoba oil, hydrogenated sunflower oil, hydrogenated castor oil, hydrogenated coconut oil, hydrogenated lanolin oil and bis(1,1,1-trimethylolpropane) tetrastearate and the waxes obtained by hydrogenation of castor oil esterified with cetyl alcohol.

14. The composition as claimed in any one of claims 1 to 12, characterized in that the wax is chosen from waxes with a tack of greater than or equal to 0.7 N.s and in particular greater than or equal to 1 N.s, and a hardness of less than or equal to 3.5 MPa.

15. The composition as claimed in claim 14, characterized in that said wax is chosen from C₂₀-C₄₀ alkyl (hydroxystearyloxy)stearates.

16. The composition as claimed in any one of claims 1 to 12, characterized in that said wax is chosen from waxes with a starting melting point of greater than or equal to 45° C., especially greater than or equal to 50° C., in particular greater than or equal to 55° C. and more particularly greater than or equal to 60° C.

17. The composition as claimed in claim 16, characterized in that said wax is chosen from carnauba wax, rice bran wax, candelilla wax, ouricurry wax, montan wax, ozokerites, the waxes obtained by Fisher-Tropsch synthesis, hydrogenated jojoba oil, bis(1,1,1-trimethylolpropane) tetrabehenate, the waxes obtained by catalytic hydrogenation of olive oil esterified with stearyl alcohol, microcrystalline waxes and polyethylene waxes.

18. The composition as claimed in any one of the preceding claims, characterized in that the total wax content is from 10% to 70%, especially from 15% to 65%, and in particular from 20% to 60%, indeed from 25% to 55% by weight relative to the total weight of the composition.

19. The composition as claimed in any one of the preceding claims, characterized in that it also comprises at least one polymer that is soluble in said nonaqueous solvent medium and that has at least one crystallizable portion.

20. The composition as claimed in claim 19, characterized in that said polymer has a molar mass ranging from 200 to 1 000 000 g/mol, in particular from 500 to 500 000 g/mol and more particularly from 1000 to 300 000 g/mol.

21. The composition as claimed in either of claims 19 and 20, characterized in that said crystallizable portion represents at least 5%, in particular at least 10% and not more than 50%, and more particularly from 30% to 50%, by weight relative to the total weight of said polymer.

22. The composition as claimed in any one of claims 19 to 21, characterized in that said polymer is chosen from copolymers of linear and saturated C₁₂ to C₃₀ alkyl (meth)acrylates and of linear C₄ to C₁₀ or branched, cyclic and/or unsaturated C₄ to C₃₀ alkyl (meth)acrylates, copolymers of vinyl esters containing linear and saturated C₁₂ to C₃₀ alkyl groups and of vinyl esters containing linear C₄ to C₁₀ or branched, cyclic and/or unsaturated C₄ to C₃₀ alkyl groups, polycondensates of polyamide type resulting from the condensation between (α) at least one acid chosen from dicarboxylic acids containing at least 32 carbon atoms and (β) an alkylenediamine, said polycondensate comprising at least one carboxylic acid end group esterified or amidated with at least one linear and saturated monoalcohol or one linear and saturated monoamine containing from 12 to 30 carbon atoms, and lipophilic polyester polycondensates whose ends are esterified with a crystallizable acid or alcohol consisting of a saturated linear C₁₂ to C₃₀ carbon-based chain.

23. The composition as claimed in any one of claims 19 to 22, characterized in that said polymer is chosen from vinyl acetate/vinyl stearate, vinyl acetate/allyl stearate, vinyl acetate/ethylene and ethylenediamine/stearyl dilinoleate copolymers, block copolymers of hydrogenated butadiene/isoprene and poly(12-hydroxystearic acid) which is esterified at one of its ends with stearic acid.

24. The composition as claimed in any one of claims 19 to 23, characterized in that said polymer is present in a content ranging from 0.01% to 30%, especially from 0.1% to 20% and in particular from 1% to 10% by weight relative to the total weight of the composition.

25. The composition as claimed in any one of the preceding claims, characterized in that it also comprises at least one film-forming polymer.

26. The composition as claimed in any one of the preceding claims, characterized in that it also comprises at least one dyestuff.

27. The composition as claimed in any one of the preceding claims, characterized in that it also comprises at least one filler.

28. The composition as claimed in any one of the preceding claims, characterized in that it also comprises at least one cosmetically acceptable additive chosen from antioxidants, preserving agents, fragrances, neutralizers, plasticizers, fibers, gelling agents and cosmetic active agents, and mixtures thereof.

29. The composition as claimed in any one of the preceding claims, characterized in that it has a flow threshold τ_{c}, measured by oscillating rheology (γ=1 Hz), ranging from 10 to 200 Pa and especially from 20 to 100 Pa.

30. A process for preparing a composition as defined according to any one of claims 1 to 29, characterized in that it comprises at least the continuous blending of at least one wax, by passing from a temperature above the melting point of said wax to room temperature with continuous cooling.

31. The process as claimed in claim 30, characterized in that it uses a continuous twin-screw blender.

32. The process as claimed in claim 30 or 31, characterized in that at least one nonaqueous solvent is added either prior to said blending or in the course of said blending.

33. The process as claimed in any one of claims 30 to 32, characterized in that at least one polymer that is soluble in a nonaqueous solvent and that has a crystallizable portion, as defined in any one of claims 19 to 24, is added prior to said blending.

34. A process for preparing a composition as defined according to any one of claims 1 to 29, characterized in that it comprises at least one step of dispersing at least one wax in the form of particles between 0.5μm and 30μm in diameter in at least one nonaqueous solvent, said solvent being at a temperature below the melting point of said wax in particle form.

35. The process as claimed in claim 34, characterized in that the dispersion is performed at room temperature.

36. The process as claimed in claim 34 or 35, characterized in that said size is from 1 to 20 μm and in particular from 5 to 10 μm.

37. The process as claimed in any one of claims 34 to 36, characterized in that said wax is chosen from carnauba wax, synthetic wax, waxes consisting of a mixture of carnauba wax and of polyethylene wax, waxes consisting of a mixture of carnauba wax and synthetic wax, polyethylene waxes and polytetrafluoroethylene waxes.

38. The process as claimed in any one of claims 34 to 37, characterized in that at least one wax as defined according to any one of claims 12 to 17 is introduced beforehand in molten form in said solvent, and the mixture thus obtained is then allowed to cool with stirring or is blended until it is at a temperature at least below the melting point of said wax in particle form.

39. The process as claimed in any one of claims 32 to 38, characterized in that said nonaqueous solvent is as defined in any one of claims 4 to 8.

40. The process as claimed in any one of claims 34 to 39, characterized in that said nonaqueous solvent is in a mixture with at least one polymer that is soluble in said nonaqueous solvent and that has at least one crystallizable portion, as defined in any one of claims 19 to 24.

41. A process for making up keratin fibers, characterized in that a composition as defined in any one of claims 1 to 29 or as obtained via a process as defined in any one of claims 30 to 40 is applied to said keratin fibers, especially the eyelashes.
